(19) United States
(12) Patent Application Publication
(10) Pub. No.: US 2003/0032994 A1
(43) Pub. Date: Feb. 13, 2003

(54) Apparatus and method for joint surgery

(76) Inventor: Douglas K. Dew, Palm Coast, FL (US)

Correspondence Address: BEUSSE, BROWNLEE, BOWDOIN & WOLTER, P. A., 390 NORTH ORANGE AVENUE, SUITE 2500, ORLANDO, FL, 32801, US

(21) Appl. No.: 10/209,168

(22) Filed: Jul. 30, 2002

Related U.S. Application Data
(60) Provisional application No. 60/308,771, filed on Jul. 30, 2001.

(51) Int. Cl. A61N 5/067; A61N 1/05

(52) U.S. Cl. 607/89; 607/101; 607/102; 607/105; 607/113; 607/96

(57) ABSTRACT

Apparatuses for controlling the surgical tissue heating process. An irrigation fluid pump provides both positive and negative pressure to allow the surgeon to accurately determine the condition of the tissue under both pressurized and non-pressurized states. The temperature of the irrigation fluid is monitored to ensure that the tissue is not overheated. The heating probe tip is supplied with a sleeve to control the heat profile during the surgery.

This application claims the benefit of Provisional Patent Application No. 60/308,771, filed on Jul. 30, 2001.

### FIELD OF THE INVENTION

The present invention relates generally to joint surgery and more particularly to advantageous surgical apparatuses for shoulder surgery, including thermal capsulorrhaphy.

### BACKGROUND OF THE INVENTION

Arthroscopy is a surgical procedure that allows the physician to visually examine the interior of a joint in the body. During an arthroscopy procedure, the physician inspects the joint surfaces and the surrounding soft tissues, such as ligaments connecting bone to bone and cartilage covering the ends of the bones at the joints. This procedure can be used to diagnose a joint problem, perform surgery that repairs a joint problem, monitor a disease, collect a tissue sample for laboratory analysis or determine the effectiveness of a treatment. Arthroscopy is commonly performed on the knee, shoulder, ankle, hip, elbow, and wrist.

During arthroscopy a thin viewing scope (an arthroscope) is inserted into the joint through a small incision in the skin. The arthroscope comprises a light source and a video camera for capturing images for display on a video monitor. These magnified images provide a clear picture of the joint for sue by the physician during surgery. If surgery is performed, additional instruments are inserted into the joint through other small incisions.

Arthroscopy may be used to perform surgery that repairs a joint problem, such as a bone repair by shaving the bone or by removing calcium deposits or bone spurs. Tom or displaced soft tissue such as ligaments and cartilage can also be repaired arthroscopically. Ligaments can be cut (cutting or releasing a ligament allows more space for other structures) or repaired by reconstruction. Scar tissue or an area of joint lining or synovium that is inflamed can be removed.

Typically, during arthroscopic surgery a tourniquet is applied and inflated to restrict blood flow to the joint. The physician makes a small incision about 0.25 in. (0.64 cm) near the joint. Before inserting the arthroscope, the joint may be injected with a saline solution to expand the surgical site. Also, during the procedure the joint can may be irrigated with solution to clear out debris or blood in the joint and improve the surgeon's view.

One application for arthroscopic surgery is the repair of a shoulder dislocation or subluxation. The shoulder is one of the most complex joints in the body and therefore more prone to dislocation than most other body joints. The shoulder joint is illustrated in the cut-away view of FIG. 1. The rounded head 8 of the humerus 10, fits into a shallow socket 12 (also referred to as the glenoid cavity) located in the upper part of the scapula 14, just above the clavicle or shoulder blade 16. The diameter of the humeral head 8 is nearly twice the diameter of the socket 12 into which the head 8 sets. The proportions of these two elements are similar to a golf ball resting upon a tee. Thus the head 8 is held only loosely within the socket 12, allowing for a wide range of motion for the shoulder joint, but the joint stability is therefore sacrificed. The humerus 10 is held within the socket 12 by a soft tissue synovial capsule 18. Under normal conditions, the synovial capsule 18 is large and loose, but provides sufficient support to restrain the head 8 in the in the socket 12.

The shoulder joint is further stabilized by fibrous ligaments that lie within the capsule 18 and by the muscles and tendons (not shown in FIG. 1) that also allow rotation of the arm. The edge of the socket 12 is rimed by a labrum 20, a fibrocartilage extension of the socket 12, that doubles the depth of the socket 12 and contributes to shoulder stability. When working properly, this ball-and-socket arrangement of the shoulder allows the arm to move in most directions, including an arc of almost 360° as shown in FIG. 2.

Shoulder instability is defined as a clinical syndrome occurring when the shoulder is sufficiently loose to produce perceptible symptoms. FIG. 3A illustrates a normal shoulder and FIG. 3B illustrates a shoulder dislocation where the humerus 10 is not contained within the socket 12. Subluxation, a form of dislocation, occurs when the bones of the joint (i.e., the humerus 10 and the socket 12) are misaligned but remain intact.

Most shoulder dislocations are either anterior or posterior. It has been observed that athletes who perform overhead actions, such as volleyball, swimming, throwing and tennis suffer shoulder pain resulting from shoulder instability. A current hypothesis suggests that repetitive shoulder movement at the extreme range of motion leads to gradual stretching of the anterior region of the capsule 18 and tightening of the posterior region. Since ligaments function in tension, they provide little stabilizing effect when lax. Thus the head 8 tends to shift in the anterior direction due to the unbalanced forces to which it is subjected. Restoration of the capsular length and thereby capsular tension is crucial to rehabilitating and preventing anterior shoulder instability.

Non-surgical treatments for shoulder instability include administration of anti-inflammatory drugs, immobilization and physical therapy. To some extent, the capsular length can be restored by specific stretching and mobilising of the shoulder joint. Known surgical procedures include Bankart repairs, SLAP repairs, and rotator cuff repairs.

Capsular shift procedures include several arthroscopic and open surgical techniques to restore capsular balance. One technique for correcting anterior capsular shift reduces capsular slack by making a “T-shaped” cut through the capsule, overlapping the anterior and inferior margins of the incision, and suturing the overlapped regions. Capsular shrinkage can also be achieved by applying heat to the loose capsule causing local heating and resulting tightening or tensioning of the capsule tissue. For example, heating a capsule area to about 65 to 71° C. results in longitudinal shortening of the capsule collagen fibres (the strong inelastic protein that is the principal component of connective tissue such as tendons and ligaments) and about a 15% to 40% reduction in the capsule size. Various arthroscopic devices are available for delivering heat to the shoulder capsule.

### BRIEF SUMMARY OF THE INVENTION

As can be seen, the techniques for returning the capsule to its original length are subject to multiple variables that reduce the success rate. The present invention teaches certain apparatuses that allow the surgeon some control over these variables and thereby improve the success rate.

An irrigation fluid pump is taught that provides both positive and negative pressure, thus allowing the surgeon to evaluate the site the capsule in both a pressurized (or tensioned) and non-pressurized state to better identify the areas that require heating to tension the tissue fibers. The temperature of the tissue at the arthroscopic site is critical, and thus according to the teachings of the present invention a temperature monitor is placed at the irrigation outflow point. The temperature is monitored by the surgeon and can also be included within a feed back loop to trigger an irrigation of the site when the fluid temperature exceeds a predetermined value. The probe heating source is equipped with a sleeve to control the tip temperature and thus avoid overheating of the tissue. A shoulder traction apparatus is taught that measures the tension on the shoulder joint during surgery and also allows several degrees of freedom for correct positioning of the shoulder.

### BRIEF DESCRIPTION OF THE FIGURES

The foregoing and other features of the invention will be apparent from the following more particular description of the invention, as illustrated in the accompanying drawings, in which like reference characters refer to the same parts throughout the different figures. The drawings are not necessarily to scale, emphasis instead being placed upon illustrating the principles of the invention.

FIGS. 1, 2, 3A and 3B illustrate various views of a shoulder joint;

FIG. 4 illustrates a pressure controllable pump according to the teachings of the present invention;

FIG. 5 illustrates a fluid temperature monitor according to the teachings of the present invention;

FIG. 6 illustrates a heating probe tip sleeve according to the teachings of the present invention;

FIGS. 7 and 8 illustrate a shoulder traction apparatus according to the teachings of the present invention.

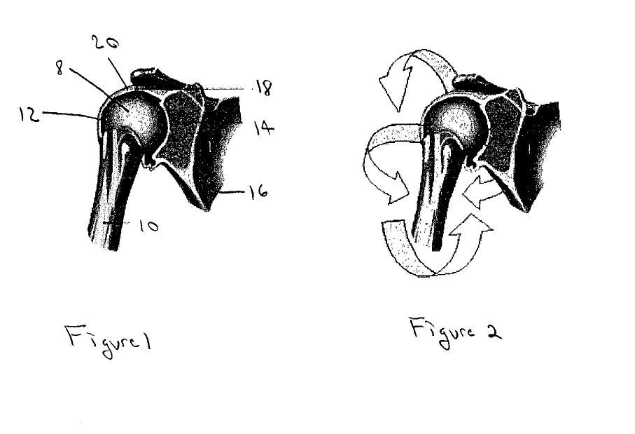

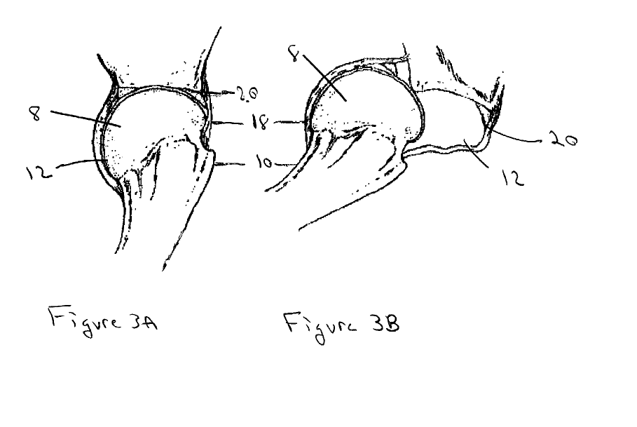

### DETAILED DESCRIPTION OF THE INVENTION

Before describing in detail the particular surgical apparatuses and techniques according to the teachings of the present invention, it should be observed that the present invention resides primarily in a novel combination of hardware elements. Accordingly, the hardware elements have been represented by conventional elements in the drawings, showing only those specific details that are pertinent to the present invention, so as not to obscure the disclosure with structural details that will be readily apparent to those skilled in the art having the benefit of the description herein.

Thermal capsulorrhaphy is the process of heating regions of the capsule to cause capsule fiber shrinkage and reduce capsule laxity. The process restores the capsular tension and the balance of the anterior and posterior capsular structures. It has been observed that capsular laxity during the arthroscopic procedure varies based on the distention employed at the arthroscopy site. For example, over-distention caused by the presence of arthroscopic fluid obliterates the variable anatomical folds of the capsule. These folds must be seen to accurately determine the sites for application of capsulorrhaphy therapy. Also, there is a direct relationship between the temperature required to contract the capsule fibers and the capsule tension. Thus higher capsule tension requires higher contraction temperatures. If the tension is excessive, it may not be possible to shrink the capsule with conventional capsulorrhaphy therapy heat sources.

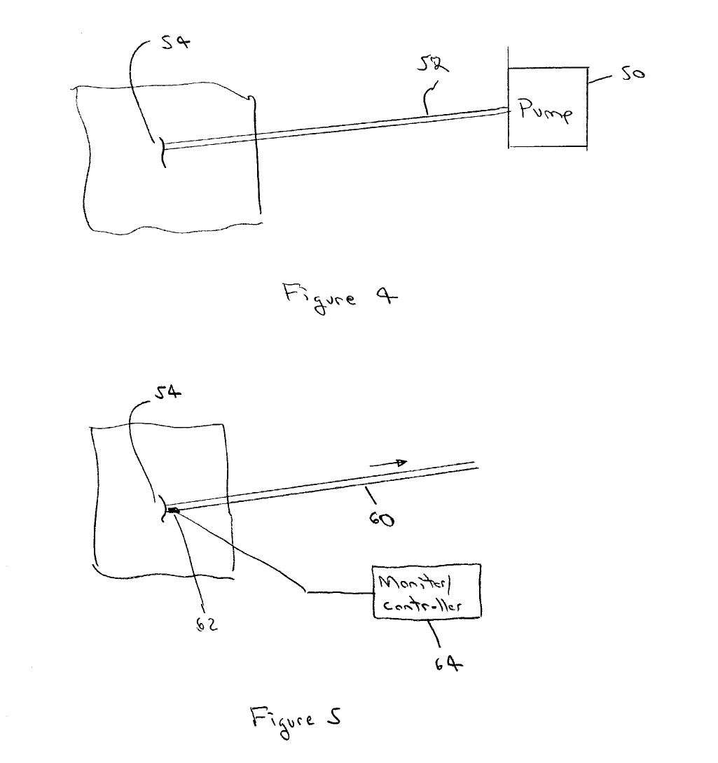

According to the teachings of the present invention, the pressure within the arthroscopic incision is controllable so that the capsule can be viewed during the application of both positive and negative pressures, improving the examination of the capsule to identify the regions where it is loose and must be contracted. This is accomplished by supplying arthroscopic fluid through a pressure controllable pump as illustrated in FIG. 4. A pump 50 provides arthroscopic irrigation fluid through a cannula 52 inserted into an arthroscopic incision 54. The pump 50 is controllable by the physician to provide fluid under positive pressure or to withdraw fluid by application of a negative pressure (i.e., a vacuum).

During the surgical procedure heat is applied to the capsule conventionally by a laser, a monopolar radio frequency (RF) source or a bipolar radio frequency source. A holmium laser operates by heating the water in the tissue. Bipolar and monopolar RF sources heat the tissue by creating molecular oscillation within the cell, with the resulting friction producing heat. Generally, the probe temperature is higher above the tip compared to the tip temperature because the heat rises from the tip. A monopolar RF source passes energy through the target tissue via a single active electrode to a dispersive pad adhered to the patient's skin. Since a complete circuit from the source to the capsule and to the pad is required, the RF electrode must be in physical contact with the capsule tissue. The bipolar source includes an active electrode for delivering energy to the tissue and a return electrode in proximity to the active electrode. Using a bipolar source, the tissue can be heated without tissue contact. Tissue damage due to excessive temperatures has been when using a bipolar probe appears to be confined to the surface area of the probe. Tissue damage for the monopolar probe tends to be more diffuse.

Irrespective of the probe type, careful control must be exercised by the physician to ensure that the tissue is not overheated, potentially producing osteonecrosis or dead tissue within the joint. Generally, cells begin to die when exposed to temperatures in excess of about 45° C., and therefore during arthroscopic surgery the temperature is kept below this threshold. But at least a minimum probe temperature must be maintained to ensure capsule shrinkage. The shrinking temperature of the capsule collagen is variable and dependent on several factors, such as age (older patients require a higher temperature to shrink the capsule tissues), the amount of capsule tension during surgery (increased pressure requires a higher shrinkage temperature), and the presence of capsule inflammation.

A nominal shrinkage temperature for the capsule tissue is about 65° C. Thus during the procedure, the surrounding tissue is maintained below about 45° C. by constantly irrigating the site while the loose regions of capsule tissue are heated to about 65° C. It is known to determine the temperature of the heater probe tip, but this value may not be indicative of and differs from the temperature of the joint tissue that is not undergoing thermal ossification during the capsulorrhaphy surgical procedure.

To ensure the correct temperatures are maintained during the procedure, according to the teachings of the present invention, the temperature of the irrigation fluid is monitored, as this value is indicative of the temperature of the normal tissue that is not to be heated. As shown in FIG. 5, in one exemplary embodiment the temperature of the irrigation fluid outflow through a cannula 60 is monitored by a temperature sensor 62, for example, comprising a thermocouple. Alternatively, a temperature sensor can be placed within the joint tissue or proximate the heater probe tip. The temperature reading is supplied to a monitor/controller 64 that can display the temperature and/or control the probe heating source. For example, the probe can be automatically turned off if the temperature reaches a predetermined value. Also, knowing the irrigation fluid temperature, the surgeon can pause the heating process and/or irrigate the site to reduce the tissue temperature.

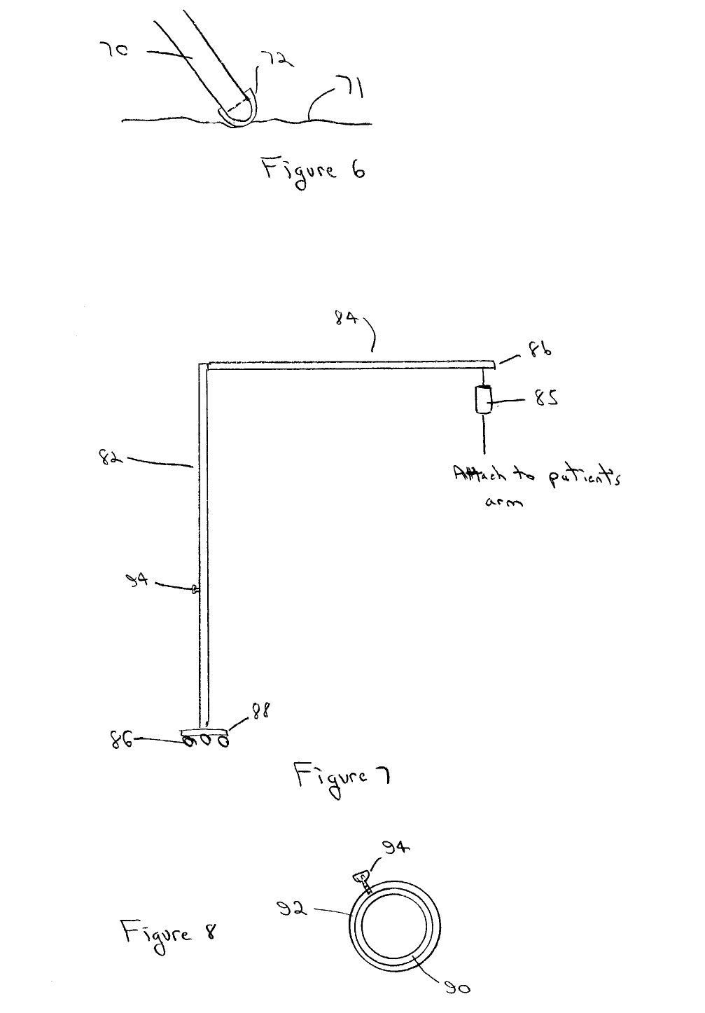

Heater probe temperatures vary along the axial direction back from the probe tip. The depth and volume of tissue heating are important parameters during the capsulorrhaphy process and these are variable among the conventional heater probes. To provide the surgeon with greater control over the application of heat from the probe, and to minimize the temperature of the tissue proximate the probe tip, according to the teachings of the present invention the probe tip is provided with a heat insulating structure. A probe tip 70 is in contact with tissue 71 as shown in FIG. 6. The probe tip 70 is insulated from the tissue 71 by an insulating sleeve 72. With the sleeve 72 in place, the surgeon can drag the tip over the tissue surface without concern that the contacted tissue will be overheated. In one embodiment the RF energy is emitted from a point about 2 mm behind the probe tip using a sleeve 72 that is about 2 mm long.

The shoulder position during surgery is critical to the success of the capsulorrhaphy process. Typical prior art shoulder positioning devices are simple multi-purpose stands with a cantilevered beam extending therefrom. With the patient in a supine position, the arm is in an abducted position and attached to the beam. The beam comprises a pulley system from which weights are hung to control the tension on the extended arm. Due to the anatomical variability presented by patients, for example, the patient's arm may be large or small, the prior art system may not provide adequate traction during surgery. Although the stand is equipped with wheels and a cranking mechanism for lowering and raising the beam, additional degrees of freedom are considered advantageous.

As shown in FIG. 7, a shoulder positioning device 80 comprises a vertical member 82 and a telescoping cantilevered beam 84 rotatably affixed thereto. The patient's arm is attached to an end point 86 of the beam 84 via a tension measuring instrument 85. Typically about 15 pounds of traction is acceptable to traction the shoulder during the capsulorrhaphy process. The telescoping feature of the beam 84 allows the end point 86 to be positioned directly above the patient's extended arm for improved traction, without repositioning the vertical member 80. The rotatable feature allows rotation of the beam 84 with respect to the vertical member 82, thus providing inferior and superior positioning with respect to the patient. In one embodiment, the shoulder positioning device 80 includes wheels 86 mounted on a platform 88 connected to the vertical member 82.

In the embodiment illustrated in FIG. 8, the vertical member is preferably circular in cross-section and includes an inner circular member 90 and an outer circular member 92. The beam 84 is attached to the inner member 90 such that the beam 84 is rotatable with respect to the outer member 92. A thumb screw 94 is threaded into the outer member 92 for urging against the inner member 90 to fix the beam 84 at the desired vertical height.

While the invention has been described with reference to preferred embodiments, it will be understood by those skilled in the art that various changes may be made and equivalent elements may be substituted for elements thereof without departing from the scope of the present invention. The scope of the present invention further includes any combination of the elements from the various embodiments set forth herein. In addition, modifications may be made to adapt a particular situation to the teachings of the present invention without departing from its essential scope. For example, the teachings can be applied to surgery performed on other body joints. Therefore, it is intended that the invention not be limited to the particular embodiments disclosed, but that the invention will include all embodiments falling within the scope of the appended claims.

What is claimed is:

1. An apparatus for controlling pressure within an arthroscopic surgery site, comprising:
a pump; and
a tube connected to said pump at a first orifice and having a second orifice at the surgical site, for carrying a fluid to the surgical site in response to the pressure exerted by said pump, wherein said pump is controllable to supply positive or negative pressure to the fluid.

2. The apparatus of claim 1 wherein the apparatus further comprises a temperature measuring device for determining the temperature of the surgery site.

3. The apparatus of claim 2 wherein the temperature measuring device is located at the second orifice for determining the temperature of the fluid.

4. A probe for supplying heat to a biological tissue, comprising:
a shaft member;
a tip attached to said shaft for emitting energy for heating tissue; and
a sleeve fitted around said tip and extending a distance axially along said shaft for providing insulation between said tip and the tissue in contact with the tip.

5. The probe of claim 4 wherein the distance that the sleeve extends along the shaft is about 2 mm.

6. The probe of claim 4 wherein the energy emitted by the tip is selected from among laser energy and radio frequency energy.

7. A surgical traction apparatus for attachment to a abducted patient's arm, said apparatus comprising:
a telescoping substantially vertical member having an upper attachment point;
a telescoping cantilevered member having first and second ends, wherein said first end is rotatably attached to said upper attachment point;
a tension measuring instrument attached between said second end and the patient's arm for measuring the tension applied to the arm.

8. The surgical traction apparatus of claim 7 further comprising a first locking device for fixing the telescoping vertical member in the preferred position and a second locking device for fixing the telescoping cantilevered member in the preferred position.

9. The apparatus of claim 7 wherein the vertical telescoping vertical member comprises an outer tubular member concentrically oriented with an inner tubular member, such that the telescoping action is provided by withdrawing said inner tubular member from within said outer tubular member.

10. The apparatus of claim 7 wherein the telescoping cantilevered member comprises an outer tubular member concentrically oriented with an inner tubular member, such that the telescoping action is provided by withdrawing said inner tubular member from within said outer tubular member.
